(19) United States
(12) Patent Application Publication
(10) Pub. No.: US 2022/0047211 A1
(43) Pub. Date: Feb. 17, 2022

(54) Sleep Quality Monitoring System for the Middle-aged and the Elderly

(71) Applicants: Xinghong Jiang, Shenzhen (CN); Wenfang Song, Shenzhen (CN); Xuechuan Duan, Shenzhen (CN); Xiang Ling, Shenzhen (CN); Yilei Chen, Shenzhen (CN)

(72) Inventors: Xinghong Jiang, Shenzhen (CN); Wenfang Song, Shenzhen (CN); Xuechuan Duan, Shenzhen (CN); Xiang Ling, Shenzhen (CN); Yilei Chen, Shenzhen (CN)

(21) Appl. No.: 16/993,287

(22) Filed: Aug. 14, 2020

(51) Int. Cl. A61B 5/00

(52) U.S. Cl. CPC A61B 5/4815; A61B 5/746; A61B 5/0022; A61B 2562/227; A61B 2562/029; A61B 2562/0247; A61B 2562/0271; A61B 2503/08

(57) ABSTRACT

The invention discloses a sleep quality monitoring system for the middle-aged and the elderly. The sleep quality monitoring system for the middle-aged and the elderly detects the sleep quality of the middle-aged and the elderly by a power socket A, an adapter A, a mobile phone APP, a cloud server, a bedside lamp, a power socket B, an adapter B, a transceiver controller, a humidity sensor, a temperature sensor, a pressure sensor, a heating pad, a signal collecting unit, a processor transmitting signal unit, a humidity sensing unit, a temperature sensing unit, an alarm unit, a preheating unit, a heating unit and a signal receiving processing unit.

### TECHNICAL FIELD

The present invention relates to the technical field of sleep quality monitoring, in particular to a sleep quality monitoring system for the middle-aged and the elderly.

### TECHNICAL BACKGROUND

People stepping into the middle-old ages pay great attention to physical and mental exercises, especially with the continuous improvement of living standards, they have growing fitness awareness and adopt more extensive fitness methods. Among them, a reasonable and normal lifestyle is the key factor to ensure human health and effective method to control most chronic diseases. Lifestyles are generally characterized by three aspects, namely, diet, activity and sleep quality. Because sleep is extremely important for the body to maintain normal physiological functions and work activities, and is closely related to health, a variety of sleep monitoring devices for the middle-aged and the elderly have appeared on the market in order to monitor the sleep quality of the middle-aged and elderly people. The specialized sleep monitoring devices on the market determine sleep quality by a comprehensive analysis and judgment through electroencephalogram, electrooculogram and mandibular electromyogram. The most commonly used sleep monitoring devices on the market mainly include two types of sleep quality monitoring devices for the middle-aged or the elderly: one of them is to determine the sleep quality of the subjects by their motion, and the other is to judge the sleep quality of the subjects by their heart rate, both of which cannot perform real-time and comfortable remote tracking and monitoring, and cannot obtain sleep quality and out-of-bed information of the elderly in time, so measures to care for the elderly and to reduce the risk cannot be taken in time. Therefore, the present invention proposes a system that can monitor the sleep quality for the middle-aged and the elderly in real time and overcome the shortcomings in the prior art.

### SUMMARY OF THE INVENTION

The purpose of the present invention is to provide a sleep quality monitoring system for the middle-aged and the elderly to solve the problems raised in the above technical background.

To achieve the above objective, the present invention provides the following technical solution: a sleep quality monitoring system for the middle-aged and the elderly comprises a power socket A, an adapter A, a mobile phone APP, a cloud server, a bedside lamp, a power socket B, an adapter B, a transceiver controller, a humidity sensor, a temperature sensor, a pressure sensor, a heating pad, a signal collecting unit, a processor transmitting signal unit, a humidity sensing unit, a temperature sensing unit, an alarm unit, a preheating unit, a heating unit and a signal receiving processing unit.

The power socket A is electrically connected to the bedside lamp through the adapter A; the mobile phone APP and the cloud server are electrically connected to the bedside lamp with a display screen through a wireless transmission module; the mobile phone APP and the cloud server are internally provided with the signal collecting unit; the power socket B is electrically connected to the transceiver controller through the adapter B; the transceiver controller is internally provided with the processor transmitting signal unit and is electrically connected to the bedside lamp through the wireless transmission module; the transceiver controller is arranged on the heating pad; one end of the humidity sensor and the temperature sensor are electrically connected to the transceiver controller, and the other end of the humidity sensor and the temperature sensor are electrically connected to the pressure sensor; the humidity sensor is electrically connected to the humidity sensing unit and the alarm unit in sequence; the temperature sensor is electrically connected to the preheating unit and the heating unit in sequence; and the signal receiving processing unit is electrically connected to the mobile phone APP and the cloud server.

Further, the input parameter of the bedside lamp is DC 8.4V 2 A, and the input of the heating pad is DC32V 4 A.

Further, the WiFi network standard is 802.11b/g/n, and the corresponding frequency is 3-4 GHz.

Further, the frequency of the wireless transceiver module is 3-4 GHz, and the corresponding transmission distance is more than 100 m.

Further, the operating temperature of the humidity sensor is 0˜60° C. and the operating humidity is 95% RH, and there are two different response times: more than 20 s for moisture absorption and more than 40 s for moisture desorption.

Further, the operating temperature of the temperature sensor is 0˜48° C. and the pressure value is more than 2.5 kg, and the corresponding pressure response time is more than 2 s.

By comparison with the prior art, the invention has beneficial effects as follows:

1. According to the sleep quality monitoring system for the middle-aged and the elderly, domestic advanced carbon film pressure sensing technology is adopted to detect in-bed, rollover and out-of-bed state of the user in sleep time, and the state data is collected and sent to a microprocessor to be converted into electrical signals, then sent to a mobile phone of the nursing staff and someone who cares for the user by the cloud server in order to inform in-bed duration, rollover frequency, out-of-bed frequency and duration; the sleep data of the user over consecutive days is acquired through a pressure sensing pad, and the sleep recording curve and the sleep quality evaluation are given through an APP interface to inform abnormal sleep phenomena in time, so that measures for improving the sleep quality of the elderly can be taken in time, such as timely verbal communication and company of family to relieve depression, loneliness and fear felt by the elderly; Through an alarm sent to the mobile phone to alert about a long-term out-of-bed state of the elderly, measures can be taken in time to avoid accidents.

2. The sleep quality monitoring system for the middle-aged and the elderly collects data of environment temperature to which the user is exposed during sleep by a temperature sensor, and then transmits radio-frequency signals to the mobile phone of the nursing staff through the cloud server. The temperature grade on the mobile phone is adjusted by the nursing staff, and then transmitted to the processor by the cloud server. Afterwards, a temperature grade adjustment signal is sent by the processor to create a comfortable sleep environment.

3. The sleep quality monitoring system for the middle-aged and the elderly collects data of environment humidity to which the user is exposed during sleep by the humidity sensor, and transmits radio-frequency signals to the mobile phone of the nursing staff through the cloud server for information. When the humidity exceeds the set standard after urination, an alarm signal is sent by the mobile phone of the nursing staff or a smart speaker to inform the nursing staff to take measures, allowing the patient to be nursed, relieving urine damage to the body and reducing the conflict between doctors and patients.

4. The sleep quality monitoring system for the middle-aged and the elderly works on the carbon fibers heating unit, also known as “the king of new materials” by adopting low voltage, and the carbon fibers emit far infrared rays that are beneficial to the human body, raise ambient temperature, promote blood circulation and accelerate metabolism.

5. According to the sleep quality monitoring system for the middle-aged and the elderly, the controller is installed in the smart speaker lamp holder to transmit and receive signals. The signals are transmitted by the cloud server in combination with the Internet of Things technology on the mobile phone APP to meet the desire of the relatives to care for the elderly, alleviate the pain of missing, enhance family affection and friendship, and harmonize the society.

### BRIEF DESCRIPTION OF THE DRAWINGS

FIG. 1 schematically illustrates the overall structure of the present invention;

FIG. 2 schematically illustrates the system framework of the present invention.

Marks in the drawings: 1—power socket A; 2—adapter A; 3—mobile phone APP; 4—cloud server; 5—bedside lamp; 6—power socket B; 7—adapter B; 8—transceiver controller; 9—humidity sensor; 10—temperature sensor; 11—pressure sensor; 12—heating pad; 13—signal collecting unit; 14—processor signal transmission unit; 15—humidity sensing unit; 16—temperature sensing unit; 17—alarm unit; 18—preheating unit; 19—heating unit; 20—signal receiving processing unit.

### DETAILED DESCRIPTION OF THE PREFERRED EMBODIMENTS

Together with the drawings in the embodiments of the present invention, the technical scheme in the embodiments of the present invention is described clearly and completely as follows. Obviously, the embodiments described are merely part of embodiments in the present invention, not all the embodiments. All the other embodiments obtained by the ordinary technical personnel in this field based on the embodiments of the present invention without paying creative work belong to the protection range of the present invention.

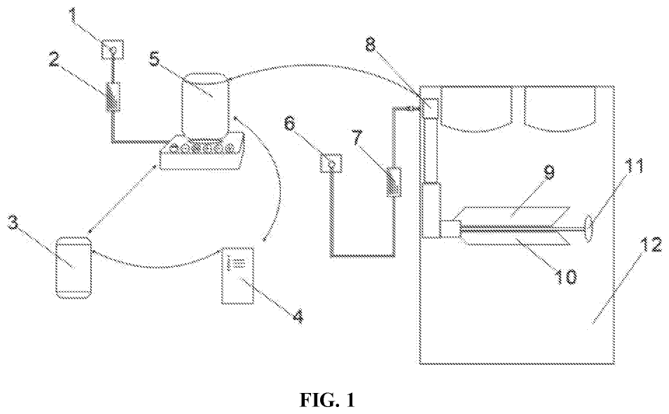

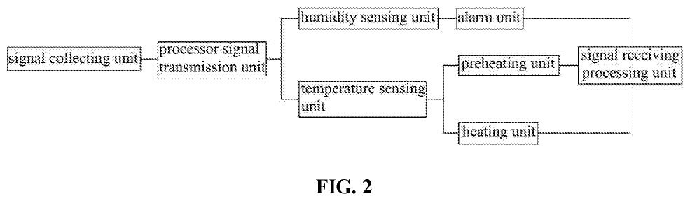

Please refer to FIG. 1-2, the present invention provides a technical solution: the sleep quality monitoring system for the middle-aged and the elderly comprises a power socket A (1), adapter A (2), mobile phone APP (3), cloud server (4), bedside lamp (5), power socket B (6), adapter B (7), transceiver controller (8), humidity sensor (9), temperature sensor (10), pressure sensor (11), heating pad (12), signal collecting unit (13), processor transmitting signal unit (14), humidity sensing unit (15), temperature sensing unit (16), alarm unit (17), preheating unit (18), heating unit (19) and a signal receiving processing unit (20).

The power socket A (1) is electrically connected to the bedside lamp (5) through the adapter A (2); the mobile phone APP (3) and the cloud server (4) are electrically connected to the bedside lamp (5) with a display screen through a wireless transmission module; the mobile phone APP (3) and the cloud server (4) are internally provided with signal collecting unit (13); the power socket B (6) is electrically connected to the transceiver controller (8) through the adapter B (7); the transceiver controller (8) is internally provided with the processor transmitting signal unit (14) and electrically connected to the bedside lamp (5) through the wireless transmission module; the transceiver controller (8) is arranged on the heating pad (12); one ends of the humidity sensor (9) and the temperature sensor (10) are electrically connected to the transceiver controller (8), and the other end of the humidity sensor (9) and the temperature sensor (10) are electrically connected to the pressure sensor (11); the humidity sensor (9) is electrically connected to the humidity sensing unit (15) and the alarm unit (17) in sequence; the temperature sensor (10) is electrically connected to the preheating unit (18) and the heating unit (19) in sequence; and the signal receiving processing unit (20) is electrically connected to the mobile phone APP (3) and the cloud server (4).

### Embodiment 1

The pressure sensor (11) is used to collect sleep data of the user over consecutive days. Through data collection from the humidity sensor (9) and the temperature sensor (10), the data is transmitted to the mobile phone APP (3) interface to give a sleep recording curve and sleep quality evaluation, so that the abnormal sleep phenomena can be found in time. In the setting interface, users can input such information as age, height and weight. The out-of-bed duration can be set, and when the sleeper leaves the bed during sleep and does not go back within the set time, the bedside lamp (5) will emit a “beep” alarm sound. Press any key to stop the alarm sound. When an out-of-bed alarm occurs, a warning message will pop up on the mobile phone APP (3) interface.

### Embodiment 2

During the intelligent period, if nobody sleeps on the heating pad (12), when the ambient temperature makes the blanket surface temperature lower than the set “preheated temperature”, the transceiver controller (8) will automatically heat the blanket surface to the set “preheated temperature” and maintain the temperature of the heating pad (12) by adjusting. In addition, if the heating pad (12) is pressed by someone during sleep, the transceiver controller (8) will heat the heating pad (12) to the set “target temperature” and maintain the temperature of the heating pad (12) by adjusting according to temperature data of the signal collecting unit (13). The temperature sensor (10) is used to collect the environment temperature at which the user is exposed during sleep, and radio frequency signals are transmitted to the mobile phone of the nursing staff through the cloud server (4). The temperature grade on the mobile phone is adjusted by nursing staff, and then sent to signal receiving processing unit (20) through the processor transmitting signal unit (14) of the cloud server (4), and then the temperature grade adjustment signal is sent by the heating unit (19).

### Embodiment 3

By touching and sliding the switch with hand, the bed-wetting alarm function can be turned on or off by the alarm unit (17). The bed-wetting alarm collects humidity data through the humidity sensor (9). When the alarm unit (17) emitting humidity alarm makes an alarm, the screen on the transceiver controller (8) will flash the humidity alarm icon and simultaneously emit “beep” alarm sound. Press any key to stop the sound. If the wet area is not processed in time, the alarm unit (17) will make an alarm again every 1 minute until the wet area is processed to dryness or the function is manually turned off to mute the alarm for processing in time.

Ordinary technical personnel in this field may make many variations, modification, replacement and changes on these embodiments without departing from the principle and spirit of the invention although the embodiment of the present invention are described, and the scope of the present invention is defined by the following claims and equivalents thereof.

1. A sleep quality monitoring system for the middle-aged and the elderly, wherein the sleep quality monitoring system for the middle-aged and the elderly comprises: a power socket A (1), an adapter A (2), a mobile phone APP (3), a cloud server (4), a bedside lamp (5), a power socket B (6), an adapter B (7), a transceiver controller (8), a humidity sensor (9), a temperature sensor (10), a pressure sensor (11), a heating pad (12), a signal collecting unit (13), a processor transmitting signal unit (14), a humidity sensing unit (15), a temperature sensing unit (16), an alarm unit (17), a preheating unit (18), a heating unit (19) and a signal receiving processing unit (20);
wherein the power socket A (1) is electrically connected to the bedside lamp (5) through the adapter A (2); the mobile phone APP (3) and the cloud server (4) are electrically connected to the bedside lamp (5) with a display screen through a wireless transmission module; the mobile phone APP (3) and the cloud server (4) are internally provided with the signal collecting unit (13); the power socket B (6) is electrically connected to the transceiver controller (8) through the adapter B (7); the transceiver controller (8) is internally provided with the processor transmitting signal unit (14) and is electrically connected to the bedside lamp (5) through the wireless transmission module; the transceiver controller (8) is arranged on the heating pad (12); the one end of the humidity sensor (9) and the temperature sensor (10) are electrically connected to the transceiver controller (8), and the other end of the humidity sensor (9) and the temperature sensor (10) are electrically connected to the pressure sensor (11); the humidity sensor (9) is electrically connected to the humidity sensing unit (15) and the alarm unit (17) in sequence; the temperature sensor (10) is electrically connected to the preheating unit (18) and heating unit (19) in sequence; and the signal receiving processing unit (20) is electrically connected to the mobile phone APP (3) and the cloud server (4).

2. The sleep quality monitoring system for the middle-aged and the elderly according to claim 1, wherein the input parameter of the bedside lamp (5) is DC 8.4V 2 A, and the input of the heating pad (12) is DC32V 4 A.

3. The sleep quality monitoring system for the middle-aged and the elderly according to claim 1, wherein the WiFi network standard is 802.11b/g/n, and the corresponding frequency is 3-4 GHz.

4. The sleep quality monitoring system for the middle-aged and the elderly according to claim 1, wherein the frequency of the wireless transceiver module is 3-4 GHz, and the corresponding transmission distance is more than 100 m.

5. The sleep quality monitoring system for the middle-aged and the elderly according to claim 1, wherein the operating temperature of the humidity sensor (9) is 0˜60° C. and the operating humidity is 95% RH, and there are two different response time: more than 20 s for moisture absorption and more than 40 s for moisture desorption.

6. The sleep quality monitoring system for the middle-aged and the elderly according to claim 1, wherein the operating temperature of the temperature sensor (10) is 0˜48° C. and the pressure value is more than 2.5 kg, and the corresponding pressure response time is more than 2 s.
